(19) United States
(12) Patent Application Publication
(10) Pub. No.: US 2013/0122554 A1
(43) Pub. Date: May 16, 2013

(54) METHOD FOR TREATING LIGNOCELLULOSIC BIOMASS

(71) Applicant: HONDA MOTOR CO., LTD., Tokyo (JP)

(72) Inventors: Masaki Ueyama, Saitama (JP); Shoji Isobe, Saitama (JP); Makoto Uda, Saitama (JP); Yuichi Itoh, Saitama (JP)

(73) Assignee: HONDA MOTOR CO., LTD., Tokyo (JP)

(21) Appl. No.: 13/670,265

(22) Filed: Nov. 6, 2012

(30) Foreign Application Priority Data
Nov. 11, 2011 (JP) 2011-248106
Oct. 26, 2012 (JP) 2012-236811

(51) Int. Cl. C12P 19/14

(52) U.S. Cl. 435/99

(57) ABSTRACT

It is intended to provide a method for treating lignocellulosic biomass, which can reliably show the completion of the course by which pretreated lignocellulosic biomass is rendered flowable and thus transportable. The method for treating lignocellulosic biomass comprises a first saccharification step of saccharifying pretreated lignocellulosic biomass with stirring using a stirring unit 7 to obtain a first saccharification product that is flowable and thus transportable and a second saccharification step of obtaining a second saccharification product from the first saccharification product, wherein the rate of change dW/dt of power W required for the stirring in the stirring unit 7 versus stirring time t falls below a predetermined value, after which the first saccharification product is transported under non-air-contact conditions from the first saccharification step to the second saccharification step.

### BACKGROUND OF THE INVENTION

1. Field of the Invention

The present invention relates to a method for treating lignocellulosic biomass.

2. Description of the Related Art

In recent years, use of blended fuel of liquid hydrocarbon (e.g., gasoline) and ethanol as automobile fuel has been studied in response to a demand for reduction in carbon dioxide emission, which is considered to be a cause of global warming. The ethanol that can be used is obtained by the fermentation of plant materials such as crops including sugarcane and corn.

The amount of carbon dioxide emitted upon combustion of the ethanol obtained from such plant materials is equal to that absorbed by the raw material plant itself, because the plant itself has already absorbed carbon dioxide through photosynthesis. This means that the carbon dioxide emission is theoretically zero-some, i.e., so-called carbon neutral effect can be obtained.

The crops such as sugarcane and corn are fundamentally food. Thus, the supply of these crops as food disadvantageously decreases if they are consumed in large amounts as raw materials for ethanol.

Accordingly, techniques have been studied to produce ethanol using lignocellulosic biomass (hereinafter, also referred to as biomass), which is not used for food, instead of the crops (e.g., sugarcane and corn) as raw materials. The lignocellulosic biomass contains cellulose and hemicellulose (hereinafter, also collectively referred to as celluloses). These celluloses can be degraded by enzymatic saccharification into sugars, such as glucose or xylose, which are then fermented to obtain ethanol.

Examples of the lignocellulosic biomass can include wood, rice straw, wheat straw, bagasse, bamboo, corn stalks, leaves, and cobs, pulp, and wastes resulting therefrom, for example, waste paper.

However, the lignocellulosic biomass is composed mainly of hemicellulose and lignin, in addition to cellulose. Since cellulose and hemicellulose are usually bound tightly to lignin, these celluloses are difficult to saccharify through direct reaction.

Thus, the conventional production of ethanol derived from lignocellulosic biomass involves: dissociating lignin from lignocellulosic biomass or swelling lignocellulosic biomass in a pretreatment chamber; saccharifying the lignocellulosic biomass thus pretreated using an enzyme in a saccharification chamber; and fermenting the obtained saccharified solution in a fermenter, followed by distillation, (see e.g., Japanese Patent Laid-Open Nos. 2006-101829 and 2008-271962).

This pretreated lignocellulosic biomass is not flowable and is thus difficult to transport from the pretreatment chamber to the saccharification chamber via a conduit or the like. For this reason, the pretreated lignocellulosic biomass is taken out of the pretreatment chamber, then housed in, for example, a container for transportation, and transported to the saccharification chamber.

Unfortunately, the pretreated lignocellulosic biomass taken out of the pretreatment chamber is contacted with the air during its transportation to the saccharification chamber and contaminated with germs. As a result, the germs consume sugars contained in the saccharified solution obtained by the saccharification, disadvantageously reducing sugar yields.

A possible approach for solving this problem and preventing the contamination with germs during the transportation is to impart flowability to the pretreated lignocellulosic biomass so as to be transportable via a conduit or the like and to transport the resulting pretreated lignocellulosic biomass under non-air-contact conditions. One possible method for imparting flowability to the pretreated lignocellulosic biomass so as to be transportable via a conduit or the like is to add water to the pretreated lignocellulosic biomass. This method, however, is not preferable because the obtained ethanol has too low concentration and requires increased energy for its distillation.

Another possible method for imparting flowability to the pretreated lignocellulosic biomass so as to be transportable is to saccharify the pretreated lignocellulosic biomass with stirring in the pretreatment chamber so that the pretreated lignocellulosic biomass is flowable and thus transportable. As described above, the pretreated lignocellulosic biomass that has been saccharified is flowable and thus transportable. Accordingly, this pretreated lignocellulosic biomass can be transported under non-air-contact conditions to the saccharification chamber via a conduit or the like and consequently, can be prevented from being contaminated with germs during the transportation. Then, the pretreated lignocellulosic biomass can be further saccharified in the saccharification chamber to complete saccharification.

For reducing cost required for the whole saccharified solution production process, it is desired that the pretreated lignocellulosic biomass should be transported to the saccharification chamber, without further stirring, immediately after the completion of the course of saccharification by which the pretreated lignocellulosic biomass is rendered flowable and thus transportable. Thus, the completion of the course by which the pretreated lignocellulosic biomass is rendered flowable and thus transportable may be shown by the time required for the stirring.

Even lignocellulosic biomass made of a single raw material differs in the state of the raw material such as lignin content, depending on conditions including harvesting areas and crop years. Inconveniently, the stirring for a predetermined time may fail to impart flowability sufficient for transportation to the pretreated lignocellulosic biomass or may stir the pretreated lignocellulosic biomass to a degree more than necessary, depending on the state of the biomass raw material.

To solve such inconvenience, an object of the present invention is to provide a method for treating lignocellulosic biomass, which can reliably show the completion of the course by which the pretreated lignocellulosic biomass is rendered flowable and thus transportable.

### SUMMARY OF THE INVENTION

In order to attain the object, the method for treating lignocellulosic biomass according to the present invention is a method for treating lignocellulosic biomass by saccharifying lignocellulosic biomass pretreated by dissociation of lignin or by swelling to obtain a saccharified solution, comprising: a pretreatment step of obtaining lignocellulosic biomass pretreated by dissociation of lignin or by swelling, in a first reactor; a first saccharification step of transferring the pretreated lignocellulosic biomass from the first reactor to a second reactor, adding a first saccharifying enzyme to the pretreated lignocellulosic biomass in the second reactor, and saccharifying the pretreated lignocellulosic biomass with stirring using a stirring unit to obtain a first saccharification product having flowability capable of being transported; and a second saccharification step of transferring the first saccharification product from the second reactor to a third reactor, adding a second saccharifying enzyme to the first saccharification product in the third reactor, and saccharifying the first saccharification product to obtain a saccharified solution as a second saccharification product, wherein after a power required for the stirring in the stirring unit gradually decreases and the rate of change of the power versus stirring time falls below a predetermined value, the first saccharification product is transported under non-air-contact conditions from the second reactor to the third reactor.

In the treatment method of the present invention, first, lignocellulosic biomass is pretreated in the pretreatment step by dissociation of lignin or by swelling in the first reactor, so as to obtain the pretreated lignocellulosic biomass.

The dissociation used herein refers to the cleavage of at least some bonds of sites at which lignin is bound to cellulose or hemicellulose in the lignocellulosic biomass. The swelling used herein refers to the entrance of a liquid to form gaps between cellulose or hemicellulose molecules constituting cellulose crystals or gaps in cellulose fibers.

Next, in the first saccharification step, the pretreated lignocellulosic biomass is transferred from the first reactor to the second reactor, the first saccharifying enzyme is added to the pretreated lignocellulosic biomass obtained by the pretreatment step in the second reactor, and the pretreated lignocellulosic biomass is saccharified with stirring using a stirring unit. As the saccharification proceeds, the pretreated lignocellulosic biomass is rendered flowable so as to be transportable via a conduit or the like. In this way, the first saccharification product is obtained.

As a result, the first saccharification product that is flowable and thus transportable can be transported easily using a transportation unit. Examples of the stirring unit that can be used include stirring blades rotated by motors, and vibration based on vibrators. Examples of the transportation unit that can be used include centrifugal pumps and Mohno pumps.

Since the flowability of the pretreated lignocellulosic biomass is enhanced with the progression of the saccharification reaction, power required for the stirring in the stirring unit gradually decreases. After the completion of the course by which the pretreated lignocellulosic biomass is saccharified into a first saccharification product that is flowable and thus transportable, the rate of change of the power versus stirring time in the stirring unit falls below a predetermined value.

Thus, power required for the stirring in the stirring unit gradually decreases and the rate of change of the power versus stirring time falls below a predetermined value, after which the first saccharification product is transported under non-air-contact conditions from the second reactor to a third reactor.

Next, in the second saccharification step, the second saccharifying enzyme is added to the transported first saccharification product in the third reactor, and the first saccharification product is saccharified to complete saccharification. In this way, a saccharified solution is obtained as the second saccharification product. The obtained second saccharification product is prevented from being contaminated with germs, because the first saccharification product is transported under non-air-contact conditions. Thus, the treatment method of the present invention can prevent sugars contained in the second saccharification product from being consumed by the germs and improve sugar yields.

As described above, according to the method for treating lignocellulosic biomass according to the present invention, power required for the stirring in the stirring unit gradually decreases and the rate of change of the power versus stirring time falls below a predetermined value, whereby the completion of the course by which the pretreated lignocellulosic biomass is rendered flowable and thus transportable can be grasped reliably. As a result, excessive stirring in the stirring unit can be prevented, and the consumption of energy required for the stirring can be reduced. Thus, energy consumption in the whole saccharified solution production process can be reduced, leading to cost reduction.

In the treatment method of the present invention, as described above, power required for the stirring in the stirring unit gradually decreases and the rate of change of the power versus stirring time fails below a predetermined value, after which the first saccharification product obtained by the first saccharification step is transported to the second saccharification step. Instead of this approach, power required for the stirring in the stirring unit gradually decreases to fall below a predetermined value, after which the first saccharification product may be transported.

In the method for treating lignocellulosic biomass according to the present invention, the more flowable first saccharification product can be transported more easily during its transportation from the second reactor to the third reactor. Thus, energy consumed by this transportation can be decreased.

However, as the flowability of the first saccharification product increases, the rate of change of the power versus stirring time becomes small. As a result, it becomes difficult to accurately determine the time for transferring the first saccharification product from the second reactor to the third reactor, on the basis of the rate of change of the power versus stirring time.

Thus, in the method for treating lignocellulosic biomass according to the present invention, preferably, the power required for the stirring in the stirring unit gradually decreases and the rate of change of the power versus stirring time falls below a predetermined value, followed by a lapse of a predetermined time, or the power required for the stirring in the stirring unit gradually decreases to fail below a predetermined value, followed by a lapse of a predetermined time, after which the first saccharification product is transported from the second reactor to the third reactor.

After a lapse of the predetermined time, the first saccharification product is rendered flowable so as to be more easily transportable. The resulting first saccharification product can be prevented from being excessively stirred in the stirring unit and can be transported easily. Thus, the total consumption of energy required for the stirring and energy required for the transportation can be reduced. As a result, energy consumption and cost in the whole saccharified solution production process can be further reduced.

In the treatment method of the present invention, the pretreatment step and the first saccharification step may be performed in separate reactors. Preferably, the pretreatment step and the first saccharification step are performed in the same reactor. In this case, it is unnecessary to transport the pretreated lignocellulosic biomass obtained by the pretreatment step from the first reactor to the second reactor. Thus, the pretreated lignocellulosic biomass can be prevented reliably from being contaminated with germs. This approach can also prevent reduction in the temperature of the pretreated lignocellulosic biomass accompanied by its transportation and can thus prevent thermal energy loss. In addition, thermal energy generated in the pretreatment step can be exploited in the first saccharification step. As a result, the time required for the pretreated lignocellulosic biomass to be flowable and thus transportable can be shortened.

In the treatment method of the present invention, as the first saccharifying enzyme, an enzyme degrading cellulose, for example, may be used. On the other hand, the second saccharifying enzyme may not be added. However, when added, the second saccharifying enzyme may be the same as the first saccharifying enzyme, or may be different therefrom. Preferably, the first saccharifying enzyme is an endo-type enzyme, which contributes to slurrying, and the second saccharifying enzyme is an enzyme mainly including an exo-type enzyme, which contributes to monosaccharification, and beta-glucosidase.

### BRIEF DESCRIPTION OF THE DRAWINGS

FIG. 1 is a system configuration diagram showing an apparatus used in a method for treating lignocellulosic biomass according to an embodiment of the present invention;

FIG. 2 is a partially cutaway perspective view showing a pretreatment chamber used in an embodiment of the present invention;

FIG. 3 is a graph showing time-dependent change in power required for stirring in the first saccharification step of the present invention;

FIG. 4 is a graph logarithmically showing the power and time required for stirring shown in FIG. 3; and

FIG. 5 is a graph showing time-dependent change in the viscosity of pretreated lignocellulosic biomass in the first saccharification step of the present invention.

### DETAILED DESCRIPTION OF THE PREFERRED EMBODIMENTS

Next, embodiments of the present invention will be described more specifically. The method for treating lignocellulosic biomass according to an embodiment of the present invention involves saccharifying lignocellulosic biomass pretreated by dissociation of lignin or by swelling to obtain a saccharified solution. The obtained saccharified solution can be used in the production of alcohols (e.g., ethanol and butanol), hydrocarbon, and resin. In an embodiment of the present invention, the saccharified solution is fermented to produce ethanol.

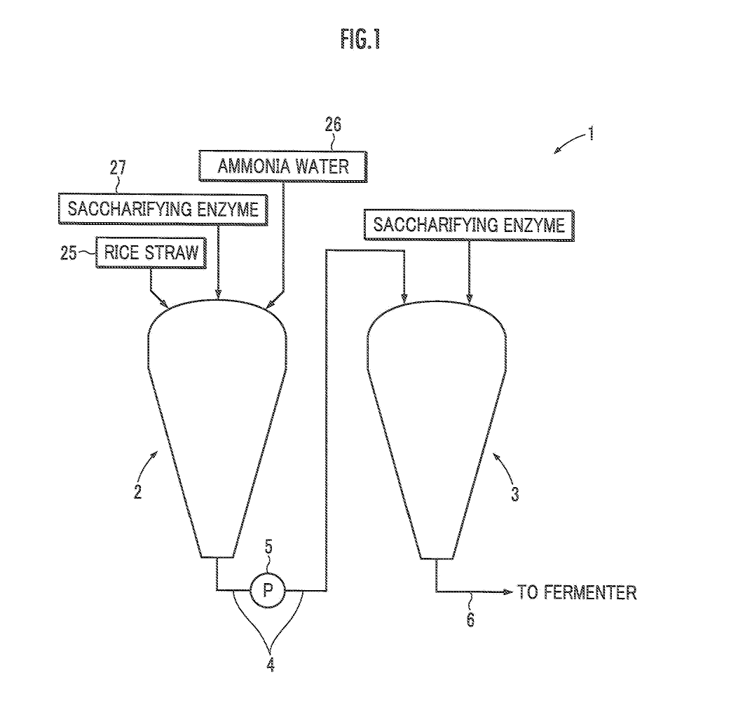

The method for treating lignocellulosic biomass according to an embodiment of the present invention is, for example, a method for treating rice straw as lignocellulosic biomass containing cellulose. This method can be performed using a treatment system 1 shown in FIG. 1. The treatment system 1 comprises a pretreatment chamber 2 and a saccharification chamber 3 which are connected to each other via a conduit 4. The conduit 4 has a transportation unit 5 which transports the contents of the pretreatment chamber 2 to the saccharification chamber 3. Examples of the transportation unit 5 that can be used include centrifugal pumps and Mohno pumps. The saccharification chamber 3 comprises a conduit 6 through which a saccharified solution is transported to a fermenter (not shown).

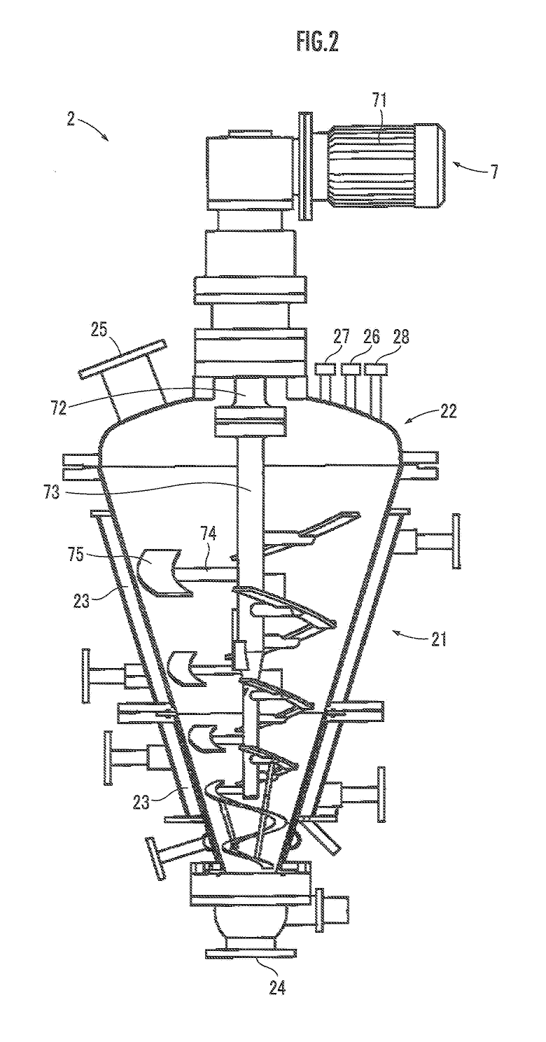

The pretreatment chamber 2, as shown in FIGS. 1 and 2, comprises an inverted cone-shaped container 21 which houses rice straw, and a lid 22 which closes the container 21. The container 21 is provided on its external side with a jacket 23 which is capable of circulating a heat medium therein and adjusting the internal temperature, and comprises, at the bottom, an outlet 24 which discharges the contents of the container 21. The outlet 24 is connected with the conduit 4.

The lid 22 is provided in its upper region with a rice straw inlet 25 from which rice straw is added, an ammonia water inlet 26 from which ammonia water is added, a saccharifying enzyme inlet 27 from which a first saccharifying enzyme degrading cellulose and hemicellulose is added, a pH adjuster inlet (not shown) from which a pH adjuster is added, and a pressure adjustment port 28 which permits adjustment of internal pressure. The pressure adjustment port 28 is connected with a pressure adjustment unit (not shown).

The lid 22 also has a stirring unit 7 which stirs the contents of the container 21. The stirring unit 7 comprises an electric motor 71 provided above the lid 22, a drive shaft 72 which is rotarily driven by the electric motor 71, and a vertical shaft 73 which is rotarily driven by the drive shaft 72. The vertical shaft 73 hangs downward in the container 21 and comprises an arm 74 which extends horizontally from the vertical shaft 73, and a stirring blade 75 attached to the end of the arm 74 to have a predetermined angle.

The stirring unit 7 can stir the contents of the container 21 via the stirring blade 75 by the electric motor 71-driven rotation of the drive shaft 72 and the vertical shaft 73.

In the method for treating lignocellulosic biomass according to an embodiment of the present invention, first, rice straw crushed in advance into a predetermined size is added into the container 21 from the rice straw inlet 25.

Next, in the pretreatment step, the stirring unit 7 stirs the rice straw in the container 21 by the rotation of the stirring blade 75, while ammonia water is added into the container 21 from the ammonia water inlet 26 to obtain a substrate mixture of the rice straw mixed with the ammonia water. Next, with stirring by the stirring unit 7 maintained, a heat medium is circulated in the jacket 23 to keep the substrate mixture for heating at a predetermined temperature for a predetermined time in the container 21. Then, ammonia contained in the rice straw can be dissipated to obtain rice straw pretreated by dissociation of lignin or by swelling.

According to an alternative embodiment, hydrothermal treatment may be performed in the pretreatment step. Specifically, water is added to the substrate mixture in the container 21 to have a predetermined water content. With the container 21 hermetically sealed, the substrate mixture is heated with stirring and kept at a predetermined temperature for a predetermined time. Then, the pressure adjustment port 28 is opened to decrease the temperature of the substrate mixture. Further, as an alternative embodiment, acid treatment may be performed in the pretreatment step, by adding acid such as dilute sulfuric acid to the substrate mixture in the container 21. Further alternatively, a steam treatment may be performed in the pretreatment step, in which steam is added with the container 21 hermetically sealed, and the substrate mixture is held at a predetermined temperature for a predetermined time, may be performed.

Next, the first saccharification step is performed using the same pretreatment chamber 2 as that used in the pretreatment step. In this first saccharification step, first, a pH adjuster is added from the pH adjuster inlet, together with water in an amount necessary for enzyme reaction, to the pretreated rice straw obtained by the pretreatment step, while a first saccharifying enzyme degrading cellulose and hemicellulose is added from the enzyme inlet 27.

Examples of the first saccharifying enzyme degrading cellulose and hemicellulose that can be used include: GC220, Accellerase 1000, Accellerase 1500, Accellerase XC, Accellerase XY, and Accellerase 1500 (distributor for all: Genencor Kyowa Co., Ltd.); Acremonium Cellulose (manufactured by Meiji Seika Pharma Co., Ltd.); and Cellic CTec and Cellic HTec (all manufactured by Novozymes).

At this time, a heat medium is circulated in the jacket 23 to keep at a predetermined temperature for a predetermined time, the pretreated rice straw containing the pH adjuster and the first saccharifying enzyme in the container 21. As a result, saccharification reaction proceeds, through which cellulose and hemicellulose contained in the pretreated rice straw are hydrolyzed by the action of the first saccharifying enzyme to form sugars.

In the saccharification reaction, first, cellulose polymer chains are fragmented, and polymer fragments with lower molecular weight are formed. As a result of the pretreatment, the cellulose or hemisellulose polymer chains are exposed, or have space between the cellulose and cellulose that permits entrance of the saccharifying enzyme. As a result of proceeding of the fragmenting of the polymer chain by the action of the saccharifying enzyme, the flowability of the pretreated lignocellulosic biomass is sharply enhanced to form the first saccharification product.

As the saccharification reaction further proceeds, the polymer fragments contained in the first saccharification product are further fragmented, so that the flowability of the first saccharification product is enhanced further.

After the completion of the saccharification reaction, a saccharified solution is formed as a second saccharification product whose components are mainly sugars such as oligosaccharides, disaccharides, or monosaccharides.

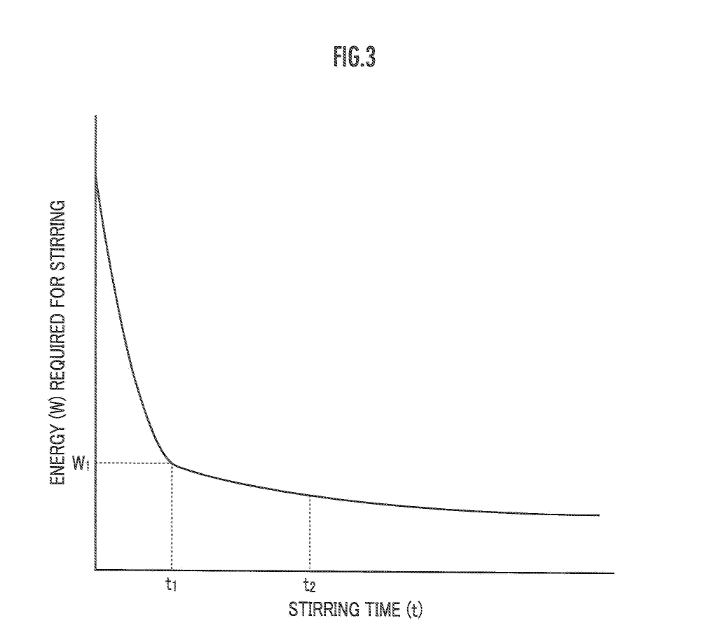

Next, the relationship between the progression of the saccharification reaction and change in power required for the stirring in the stirring unit 7 is shown in FIG. 3. As shown in FIG. 3, power required for the stirring in the stirring unit 7 gradually decreases with the progression of the saccharification reaction. Then, the pretreated lignocellulosic biomass in the container 21 reaches the state where the cellulose polymer chains are fragmented, and polymer fragments with lower molecular weight are formed, after which the flowability of the pretreated lignocellulosic biomass is sharply enhanced while the power required for the stirring in the stirring unit 7 sharply decreases. As a result, the rate of change dW/dt of the power versus stirring time falls below a predetermined value when the stirring time of the stirring unit 7 is t₁ and the power is W₁. Thus, it can be confirmed that the pretreated lignocellulosic biomass in the container 21 has been saccharified into the first saccharification product that is flowable and thus transportable.

Thus, power required for the stirring in the stirring unit 7 gradually decreases and the rate of change dW/dt of the power W versus the stirring time t falls below a predetermined value, after which the first saccharification product is discharged from the outlet 24 of the container 21 and transported through the transportation unit 5 to the saccharification chamber 3 in which the second saccharification step is performed.

As described above, the rate of change dW/dt of the power W versus the stirring time t falls below a predetermined value, after which the first saccharification product is transported to the saccharification chamber 3. Instead of this approach, power required for the stirring in the stirring unit 7 gradually decreases to fall below a predetermined value, after which the first saccharification product may be transported. In this case, the predetermined value can be set by measuring in advance change in the power required for the stirring in the stirring unit 7 and determining a value that can show that the pretreated lignocellulosic biomass in the container 21 has been saccharified into the first saccharification product that is flowable and thus transportable.

Alternatively, the rate of change dW/dt of the power W versus the stirring time t falls below a predetermined value, followed by a lapse of a predetermined time, or the power required for the stirring in the stirring unit gradually decreases to fall below a predetermined value, followed by a lapse of a predetermined time, i.e., the stirring time reaches t₂, the first saccharification product in the container 21 reaches the state where the polymer fragments contained in the first saccharification product are further fragmented. As a result, the flowability of the first saccharification product is further enhanced so as to be more easily transportable to the saccharification chamber 3, while the power required for the stirring in the stirring unit 7 no longer decreases.

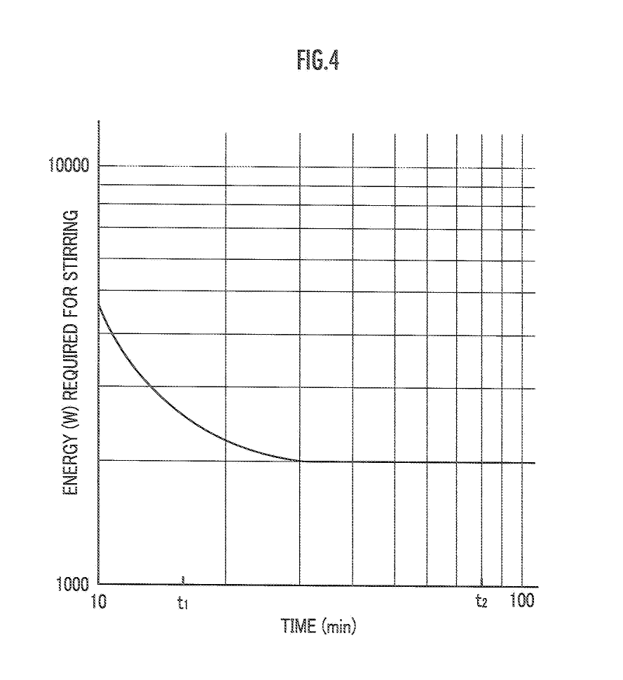

Regarding the state of the power required for the stirring shown in FIG. 3, the power required for the stirring in the stirring unit 7 still continuously changes as is shown in FIG. 4, when logarithmically plotting the power W required for the stirring in the stirring unit and the stirring time t shown in FIG. 3. Further, after continuing stirring with the temperature maintained, a steady state at which the power required for the stirring is no longer changed is achieved at time t₂. This means that a continuation of stirring after the stirring time t₂ merely makes the stirring excessive and is hardly beneficial to the transportation of the first saccharification product to the saccharification chamber 3.

Thus, the rate of change dW/dt of the power W versus the stirring time t falls below a predetermined value, followed by a lapse of a predetermined time, or the power required for the stirring in the stirring unit 7 gradually decreases to fall below a predetermined value, followed by a lapse of a predetermined time (i.e., the stirring time reaches t₂), after which the first saccharification product may be transported to the saccharification chamber 3.

The resulting first saccharification product can be prevented from being excessively stirred in the stirring unit 7 and can be transported easily. Thus, the total consumption of energy required for the stirring and energy required for the transportation can be reduced. As a result, energy consumption in the whole ethanol production process can be further reduced.

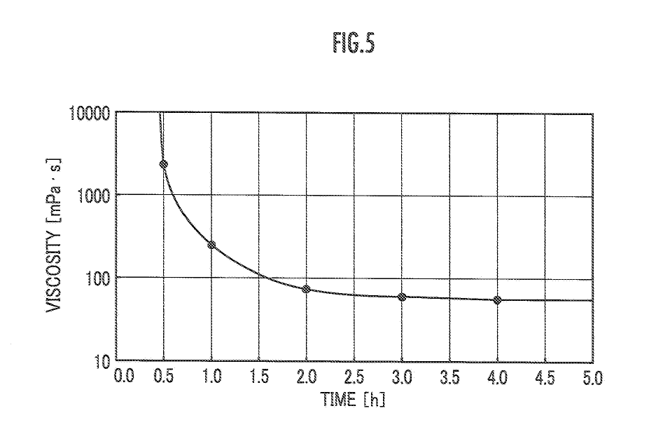

The power required for the stirring in the stirring unit 7 may vary depending on the state (e.g., maintenance) of the apparatus. Thus, as shown in FIG. 5, time-dependent change in the viscosity of the pretreated lignocellulosic biomass (or the first saccharification product) may be determined in advance to relate the viscosity to the power.

As a result, the power required for the stirring in the stirring unit 7, even when varying, can be calculated from the viscosity to thereby correctly determine whether or not the rate of change dW/dt of the power W versus the stirring time t has fallen below a predetermined value or whether or not the power required for the stirring in the stirring unit 7 has gradually decreased to fall below a predetermined value.

Alternatively, torque generated by the pretreated lignocellulosic biomass (or the first saccharification product) with respect to the stirring unit 7 and power consumption therein may be determined in advance instead of the viscosity to thereby determine, from the power consumption, whether or not the rate of change dW/dt of the power W versus the stirring time t has fallen below a predetermined value or whether or not the power required for the stirring in the stirring unit 7 has gradually decreased to fall below a predetermined value.

Next, the transported first saccharification product is subjected to the second saccharification step using the saccharification chamber 3. Meanwhile, rice straw is newly added to the pretreatment chamber 2 and subjected to the pretreatment step and the rust saccharification step.

In the second saccharification step, a saccharified solution is formed as a second saccharification product whose components are mainly sugars such as oligosaccharides, disaccharides, or monosaccharides. The second saccharifying enzyme may be the enzyme different from the first saccharifying enzyme, or may be the same enzyme. Or, the second saccharifying enzyme may not be added. Preferably, the first saccharifying enzyme is an endo-type enzyme, which contributes to slurrying, and the second saccharifying enzyme is an enzyme mainly including an exo-type enzyme, which contributes to monosaccharification, and beta-glucosidase.

The saccharified solution as the second saccharification product obtained by the second saccharification step is prevented from being contaminated with germs, because the first saccharification product is transported under non-air-contact conditions to the saccharification chamber 3. Thus, the treatment method of this embodiment can prevent sugars contained in the saccharified solution as the second saccharification product from being consumed by the germs and improve sugar yields.

Next, the obtained saccharified solution as the second saccharification product can be transported to the fermenter and fermented, followed by distillation to obtain ethanol.

In the treatment method of this embodiment, both the pretreatment step and the first saccharification step are performed in the pretreatment chamber 2. Thus, it is unnecessary to transport the pretreated lignocellulosic biomass obtained by the pretreatment step to other reactor. Thus, the pretreated lignocellulosic biomass can be prevented reliably from being contaminated with germs, compared with the case where the pretreatment step and the first saccharification step are performed in separate reactors. This approach can also prevent reduction in the temperature of the pretreated lignocellulosic biomass accompanied by its transportation and can thus prevent thermal energy loss. In addition, thermal energy generated in the pretreatment step can be exploited in the first saccharification step. As a result, the time required for the pretreated lignocellulosic biomass to be saccharified into the first saccharification product that is flowable and thus transportable can be shortened.

In the treatment method of this embodiment, the stirring blade 75 which is rotarily driven by the electric motor 71 is used in the stirring unit 7. Instead of this stirring blade, vibration based on vibrators, or the like may be used.

In the treatment method of this embodiment, rice straw is used as the lignocellulosic biomass. Instead of this rice straw, wood, wheat straw, bagasse, bamboo, corn stalks, leaves, and cobs, pulp, and wastes resulting therefrom, for example, waste paper, may be used.

Next, Example of the present invention will be shown.

### Example

In this Example, first, 386 kg of air-dried rice straw (lignocellulosic biomass) having a water content of 12% by mass was crushed using a cutter mill into a size that permits passage through a sieve having a mesh size of 3 mm, and then added from the rice straw inlet 25 into the container 21 of the pretreatment chamber 2 (trade name: PV Mixer, manufactured by KOBELCO ECO-SOLUTIONS Co., Ltd., internal capacity: 2000 L) shown in FIG. 2.

Next, in the pretreatment step, ammonia water having a concentration of 25% by mass with respect to the same mass as the dry mass 340 kg of the rice straw (386 kg) was added into the container 21 from the ammonia water inlet 26, while the rice straw, etc., in the container 21 was stirred using the stirring unit 7 to obtain a substrate mixture of the rice straw mixed with the ammonia water.

Next, with stirring by the stirring unit 7 maintained, a heat medium was circulated in the jacket 23 to keep the rice straw, etc., for heating at 80° C. for 8 hours in the container 21. Ammonia contained in the rice straw, etc. from which lignin was thus dissociated or which was thus swollen was dissipated to obtain pretreated rice straw. The pretreated rice straw was neither flowable nor transportable.

Next, in the first saccharification step, 5% by mass of diluted sulfuric acid was added to the pretreated rice straw in the container 21 from the pH adjuster inlet to adjust the pH of the pretreated rice straw to the range of 4 to 4.5, while 15 kg of a saccharifying enzyme (trade name: Acremonium Cellulase, manufactured by Meiji Seika Pharma Co., Ltd.) degrading cellulose and hemicellulose was added as the first saccharifying enzyme from the saccharifying enzyme inlet 27 as well as water was added to adjust the concentration of the pretreated rice straw to 26% by mass.

At this time, a heat medium was circulated in the jacket 23 to keep at 50° C. the pretreated rice straw containing the pH adjuster and the first saccharifying enzyme in the container 21, while the pretreated rice straw was stirred. As a result, saccharification reaction proceeded, through which cellulose and hemicellulose contained in the pretreated rice saw were hydrolyzed by the action of the first saccharifying enzyme to form sugars.

Power W required for the stirring in the stirring unit 7 and a stirring time t were graphed and thus obtained as shown in FIG. 3. As is evident from FIG. 3, when the power W required for the stirring in the stirring unit 7 was W₁ and the stirring time t was t₁, the pretreated lignocellulosic biomass in the container 21 reached the state where the polymer chains of the pretreated lignocellulosic biomass are fragmented, and polymer fragments with lower molecular weight are formed, and the rate of change dW/dt of the power W versus the stirring time t fell below a predetermined value.

Next, the power W required for the stirring in the stirring unit 7 was smaller than W₁ when the stirring time t was t₂. At this time, as is evident, the first saccharification product reached the state where the polymer fragments contained in the first saccharification product are further fragmented, and was rendered flowable so as to be more easily transportable.

What is claimed is:

1. A method for treating lignocellulosic biomass by saccharifying lignocellulosic biomass pretreated by dissociation of lignin or by swelling to obtain a saccharified solution, comprising:
a pretreatment step of obtaining lignocellulosic biomass pretreated by dissociation of lignin or by swelling, in a first reactor;
a first saccharification step of transferring the pretreated lignocellulosic biomass from the first reactor to a second reactor, adding a first saccharifying enzyme to the pretreated lignocellulosic biomass in the second reactor, and saccharifying the pretreated lignocellulosic biomass with stirring using a stirring unit to obtain a first saccharification product having flowability capable of being transported; and
a second saccharification step of transferring the first saccharification product from the second reactor to a third reactor, adding a second saccharifying enzyme to the first saccharification product in the third reactor, and saccharifying the first saccharification product to obtain a saccharified solution as a second saccharification product, wherein
after a power required for the stirring in the stirring unit gradually decreases and the rate of change of the power versus stirring time falls below a predetermined value, the first saccharification product is transported under non-air-contact conditions from the second reactor to the third reactor.

2. The method for treating lignocellulosic biomass according to claim 1, wherein
after the power required for the stirring in the stirring unit gradually decreases and the rate of change of the power versus stirring time falls below a predetermined value, followed by a lapse of a predetermined time, the first saccharification product is transported from the second reactor to the third reactor.

3. The method for treating lignocellulosic biomass according to claim 1, wherein
the pretreatment step and the first saccharification step are performed using the same reactor.

4. The method for treating lignocellulosic biomass according to claim 1, wherein
the first saccharifying enzyme is an enzyme degrading cellulose and hemicellulose.

5. The method for treating lignocellulosic biomass according to claim 1, wherein
the first saccharifying enzyme is an endo-type enzyme, which contributes to slurrying, and
the second saccharifying enzyme is an enzyme mainly including an exo-type enzyme, which contributes to monosaccharification, and beta-glucosidase.

6. A method for treating lignocellulosic biomass by saccharifying lignocellulosic biomass pretreated by dissociation of lignin or by swelling to obtain a saccharified solution, comprising:
a pretreatment step of obtaining lignocellulosic biomass pretreated by dissociation of lignin or by swelling in a first reactor;
a first saccharification step of transferring the pretreated lignocellulosic biomass from the first reactor to a second reactor, adding a first saccharifying enzyme to the pretreated lignocellulosic biomass in the second reactor, and saccharifying the pretreated lignocellulosic biomass with stirring using a stirring unit to obtain a first saccharification product having flowability capable of being transported; and
a second saccharification step of transferring the first saccharification product from the second reactor to a third reactor, adding a second saccharifying enzyme to the first saccharification product in the third reactor, and saccharifying the first saccharification product to obtain a saccharified solution as a second saccharification product, wherein
after a power required for the stirring in the stirring unit gradually decreases to fall below a predetermined value, the first saccharification product is transported under non-air-contact conditions from the second reactor to the third reactor.

7. The method for treating lignocellulosic biomass according to claim 6, wherein
after the power required for the stirring in the stirring unit gradually decreases to fall below a predetermined value, followed by a lapse of a predetermined time, after which the first saccharification product is transported from the second reactor to the third reactor.

8. The method for treating lignocellulosic biomass according to claim 6, wherein
the pretreatment step and the first saccharification step are performed using the same reactor.

9. The method for treating lignocellulosic biomass according to claim 6, wherein
the first saccharifying enzyme is an enzyme degrading cellulose and hemicellulose.

10. The method for treating lignocellulosic biomass according to claim 6, wherein
the first saccharifying enzyme is an endo-type enzyme, which contributes to slurrying, and
the second saccharifying enzyme is an enzyme mainly including an exo-type enzyme, which contributes to monosaccharification, and beta-glucosidase.
